(19) United States
(12) Patent Application Publication
(10) Pub. No.: US 2009/0298171 A1
(43) Pub. Date: Dec. 3, 2009

(54) Method and Device for Treating or Selecting Cells

(76) Inventors: Jozef Bizik, Bratislava (SK); Ari Lasse Juhani Harjula, Helsinki (FI); Esko Markus Kankuri, Helsinki (FI)

Correspondence Address: BIRCH STEWART KOLASCH & BIRCH, PO BOX 747, FALLS CHURCH, VA, 22040-0747, US

(21) Appl. No.: 12/295,599

(22) PCT Filed: Mar. 30, 2007
(86) PCT No.: PCT/FI2007/050179

§ 371 Date: Nov. 19, 2008

Related U.S. Application Data
(60) Provisional application No. 60/787,660, filed on Mar. 31, 2006.

(51) Int. Cl. C12N 5/06; C12M 1/34

(52) U.S. Cl. 435/373; 435/287.1

(57) ABSTRACT

The present invention is directed to a method of treating cells by co-culturing them with activated fibroblasts in order to regulate the growth and/or status of the cells. Fibroblasts are activated by culturing the cells under conditions that induce the cells to adhere to each other to form multicellular aggregates or spheroids. The present invention also provides a device for selecting cells from cell samples, such as bone marrow aspirate, the device comprises said multicellular aggregates.

The present invention is directed to a method of treating cells by co-culturing them with activated fibroblasts in order to regulate the growth and/or status of the cells. Fibroblasts are activated by culturing the cells under conditions that induce the cells to adhere to each other to form multicellular aggregates or spheroids. The present invention also provides a device for selecting cells from cell samples, such as bone marrow aspirate, the device comprises said multicellular aggregates.

### BACKGROUND OF THE INVENTION

Control over cell differentiation and proliferation requires complex paracrine and autocrine signals to be orchestrated both spatially and temporally. The majority of these cues originate from mesenchymal cells surrounding the immature precursor cells. In mesenchymal tissues, fibroblasts are ubiquitous sentinel cells (Mayani et al, 1992; Torok-Storb et al, 1999) that modulate series of developmental and pathologic conditions ranging from cell differentiation and organogenesis to inflammation and cancer (Bhowmick et al, 2004). Being the major stromal cellular constituents, fibroblasts play a dominant role in control over differentiation and proliferation of hematopoietic precursors (Greenberg et al, 1988; Kubota et al, 1988; Wang & Sullivan, 1992). In culture, fibroblasts support proliferation of both normal and malignant hematopoietic precursors (Rogalsky et al, 1991; Bendall et al, 1994; Buske et al, 1994; Bradstock et al, 1996). They are a rich source of several factors governing hematopoiesis (Silzle et al, 2004; Smith et al, 1997). Proper maturation of leukocytes requires strict control over the prevailing proliferative activity of the immature blasts, and is achievable only by a reciprocal complex interaction with their surrounding mesenchyme (Tavassoli & Friedenstein, 1983; Youn et al, 2000) Leukemic cells have, however, lost their ability to translate these control signals properly, and remain undifferentiated and intensely proliferating (Griffin & Löwenberg, 1986; Dührsen & Hossfeld, 1996).

By enhancing fibroblast propensity for homotypic cell-cell contacts, we recently revealed a new type of biological process triggered in dermal fibroblasts (Bizik et al., 2004). These cells, characterized by high expression of specific genes such as cyclooxygenase-2 and by increased proteolytic activity such as α-enolase-mediated plasminogen activation, are committed to death, with typical morphological signs of necrosis. Based on the unique features of this distinct type of autoactivation of fibroblasts and the consequent elimination of these cells, we designated this process neinosis (Kankuri et al, 2005),

We subsequently found that these cells produced profuse amounts of hepatocyte growth factor/scatter factor (HGF/SF), and that exposure to these nemotic fibroblasts dramatically enhanced tumor cell invasiveness (Kankuri et al, 2005). We demonstrated this effect to be exclusively mediated by HGF/SF via a transient phosphorylation of the c-met proto-oncogene product, the membrane-receptor tyrosine kinase c-Met, detectable only when this receptor underwent proper processing in the tumor cells (Kankuri et al, 2005). In addition to its several multifunctional roles as a mitogen (Igawa et al, 1991), motogen (Matsumoto et al, 1995), and morphogen (Montesano et al, 1991) for cells of epithelial and mesenchymal origin, HGF/SF is a regulator of hematopoiesis, as well (Kmiecik et al, 1992).

Based on our findings on enhancement of epithelial tumor cell progression by nemotic fibroblast-derived HGF/SF (Kankuri et al, 2005), we analyzed the effect of nemosis-derived factor(s) on cells of hematologic malignancies. We now report that when cell lines differed in phenotype by expression of proteins such as c-Met, they responded to fibroblast nemesis differently. The c-Met-negative cell lines responded with discernible growth arrest, chemotaxis, and differentiation, whereas the c-Met-positive cells remained largely unresponsive.

### BRIEF DESCRIPTION OF THE DRAWINGS

FIGS. 1A, 1B and 1C. c-Met expression in leukemia cell lines, and growth characteristics of selected

cell lines subjected to nemosis.

A: Expression of the c-Met receptor in leukemia cell lines KG-1, THP-1, U-937, Jurkat, Raji, and K562. Arrow indicates position of the properly processed form of c-Met (145 kDa)

B: Proliferation kinetics of leukemia cell lines KG-1, THP-1, and U-937 with and without stimulation by nemotic fibroblast spheroids in co-culture. * p<0.05, ** p<0.01, *** p<0.001 between treatments at indicated time-points.

C: DNA histogram data and percentage of cells as divided into cell cycle G0G1, G2, and S phases of leukemia cell lines KG-1, THP-1, and U-937 with (closed bars) and without (open bars) stimulation by nemotic fibroblast spheroids in co-culture for 96 hours.

FIGS. 2A, 2B and 2C. Lentiviral vector transduction of c-Met into THP-1 cells.

A: Expression of c-Met in the leukemia cell lines KG-1, THP-1, and U-937 stimulated for 96 hours by nemotic fibroblast spheroids (+) and without stimulation (−).

B: c-Met expression kinetics in GFP-control and c-Met-transduced THP-1 cells at indicated time points.

C: Proliferation kinetics of THP-1 cells transduced with GFP-control or c-Met lentiviral vector with or without stimulation by nemotic fibroblast spheroids in co-culture. * p<0.05, ** p<0.01, *** p<0.001 between treatments at indicated time-points.

FIGS. 3A, 3B, 3C and 3D. Adherence, morphology, and chemotactic response of the leukemic cells to nemosis.

A: Percentage of cells from the total cell population adhering to culture dish after co-culture stimulation by nemotic fibroblasts as compared to unstimulated cells of leukemia cell lines KG-1, THP-1, and U-937. *** p<0.001 compared to respective control cells.

B: Morphology of adherent cells with or without stimulation by nemotic fibroblast spheroids. Cell elongation and presence of pseudopodia evident in stimulated KG-1 and TPP-1 cells whereas, after stimulation, U-937 cells retain their phenotype.

C: Expression of intercellular adhesion molecule-I (ICAM-1) in KG-1, THP-1, and U-937 cells with (+) or without (−) stimulation by nemotic fibroblast spheroids in co-culture. Increased ICAM-1 expression of the nemosis-responsive cell lines KG-1 and THP-1 associated with increased adherence to the cell culture dish.

D: Quantification and morphology of leukemia-cell (KG-1, THP-1, U-937) chemotactic movement towards fibroblast clusters in co-culture. Increased chemotactic accumulation of KG-1 and THP-1 cells visible around a nemotic spheroid, whereas U-937 cells are unresponsive. *** p<0.001 compared to U-397 cells.

FIG. 4. Time-dependence of CD86 surface antigen expression.

CD86 expression evaluated by FACS at indicated time points for leukemia cell lines KG-1, THP-1, and U-937 in co-culture with nemotic fibroblast spheroids.

FIG. 5. Device for separating cells from a patient sample.

FIG. 6. Hematopoiesis-associated cytokine release from fibroblast spheroid clusters and monolayers.

Analysis of cytokine (interleukin IL-1, IL-6, IL-8, IL-I1, granulocyte-macrophage colony-stimulating factor, GM-CSF, and leukemia inhibitory factor, LIF) production from fibroblasts cultured as spheroids or as monolayers at the corresponding time (96 hours) after spheroid formation. *** p<0.001 between groups.

FIGS. 7A and 7B. Immunoblot analysis of apoptosis-related and intracellular signaling proteins in leukemia cells.

A: Expression of apoptosis-related molecules in leukemia cell lines KG-1, THP-1, and U-937 with (+) or without (−) stimulation by nemotic fibroblast spheroids in co-culture for 96 hours. In nemosis-responsive cell lines KG-1 and TIP-1, activation-associated cleavage of caspase-3 and -8 is evident. No cleavage products of caspase-9 and reduced expression of the cleaved form of poly(ADP-ribose)polymerase (PARP) are visible. Phenotype differences between nemosis-responders and the nemosis-unresponsive cell line U-937 evident in expression levels of the pro-apoptotic Bax protein.

B: Expression of phosphorylated and total levels of mitogen-activated protein kinases (MAPK) p38, JNK, ERK1/2, and the Akt kinase in comparison with expression of the differentiation-associated Janus-kinase family JAK1, JAK2, JAK3, and TYK2 in leukemia cell lines KG-1, THP-1, and U-937, stimulated (+) or unstimulated (−) with nemotic fibroblast spheroids. Increased dephosphorylation of p38 and ERK1/2 is evident in the nemosis-responsive cell lines KG-1 and THP-1 together with increased expression of JAK1 and JAK3. The nemosis-unresponsive cell line U-937 showed no expressional differences for these proteins.

FIG. 8. Schematic model summarizing effects of nemosis-derived signals on solid and hematopoietic tumor cells.

Schematic presentation of fibroblast nemosis effects and mediators inducing invasiveness of epithelial tumor cells together with arrest of growth and induction of differentiation of leukemic cells in a target-cell phenotype-dependent manner. Dashed lines link possible biological and pathological processes with this cascade.

### DETAILED DESCRIPTION OF THE PRESENT INVENTION

The present invention is based on the discovery that clustered or aggregated fibroblasts or other mesenchymal cells, such as bone marrow mesencymal stem cells, are able to induce a cytostatic, growth inhibitory, and/or differentiating response in primary cells in patient samples and cell lines, i.e. target cells, when such clustered cells or the cytokines and growth factors produced by the aggregates are in close vicinity or in contact with target cells, or when target cells are treated with said compounds deriving from said aggregates. Therefore, the invention provides a method for treating a cell sample, or isolating, and/or enriching cells from a cell sample, said method comprising:

a) culturing fibroblast or mesenchymal cells under conditions that promote or induce the cells to contact each other so that multicellular aggregates are forned (this phenomenon is called as nemosis in the Experimental Section below); and

b) co-culturing said cell sample with said multicellular aggregates obtained from step a) in order to regulate the growth and/or status of cells in said cell sample.

Step b) may also be performed by treating said cell sample with factor(s) derived from or produced by said multicellular aggregates.

While co-culturing, there preferably is a semi-permeable barrier or membrane between said cell sample and the multicellular aggregates, said barrier permitting exchange of cytokines and growth factors but separating physically said spheroids from said cell sample. In this context, the expression “separating physically” means that the multicellular aggregates are not in direct contact with the cells in said cell sample. The pore size of said semi-permeable barrier may preferably be 0.2 to 2 μm. The material for the semi-permeable membrane can be polyethylene terephthalate, polycarbonate, mixed cellulose esters, or teflon.

Preferably, said cell sample contains mononuclear cells isolated from bone marrow aspirate(s). More preferably, said cell sample is from patient(s) with malignant disease such as leukemia.

The term “cell sample” refers herein to a sample containing cultured cells, such as cells of a known cell line, or to a biological sample, such as a patient sample, e.g. a blood sample. Other preferable patient sample is a bone marrow aspirate.

The above method may also comprise a further step of c) selecting those cells from said cell sample which responded to the co-culturing with said multicellular spheroids. Generally, the selected cells respond to the co-culturing by chemotactic movement. Preferably, said cells are selected for a therapeutic or diagnostic use.

Another embodiment of the present invention is a device for selecting cells from any sample containing living cells, see FIG. 5 for details. Said device comprises a first compartment and a second compartment, said first compartment being arranged within the second compartment, wherein said first compartment comprises multicellular spheroids of fibroblast cells or mesenchymal stem cells in a buffer, said first compartment being separated from the second compartment by a semi-permeable membrane allowing the exchange of buffer, cytokines and growth factors between the compartments; and wherein the second compartment is separated from the sample of cells by a second membrane having a pore size allowing cells to migrate across the membrane. The pore size may preferably be 3 to 8 μm. The material for the second membrane may be polyethylene terephthalate, polycarbonate, mixed cellulose esters, or teflon.

Preferably, said compartments and the cell sample are surrounded by an outer sealing membrane.

Preferably, said semi-permeable membrane does not allow the exchange of cells between said compartments.

One preferred embodiment of the invention is to use autologous fibroblasts in step a), if the cell sample is a patient sample and the cells in the sample are to be used in a therapy of said patient. However, also allogeneic fibroblasts can be used in the invention, since the activated fibroblast cells are preferably not in direct contact with the cell sample.

All publications cited in this specification are herein incorporated by reference as if each individual publication were specifically and individually indicated to be incorporated by reference. The following examples will assist those skilled in the art to better understand the invention and its principles and advantages. It is intended that these examples be illustrative of the invention and not limit the scope thereof.

### EXPERIMENTAL SECTION

### Materials and Methods

Materials—Antibodies for immunoblotting were rabbit anti-p38 antibody (Ab) (sc-535, Santa Cruz Biotechnology Inc, Santa Cruz, Calif.), mouse anti-p-p38 Tyr182 monoclonal antibody (MAb) (sc-7973), rabbit anti-JNK Ab (CST-0252, Cell Signaling Technology, Danvers, Mass.), mouse anti-p-JNK Thr183/Tyr185 MAb (sc-6254), rabbit anti-ERK1/2 Ab (sc-94), mouse anti-p-ERK1/2 Tyr204 MAb (sc-7383), rabbit anti-Akt Ab (CST-9272), rabbit anti-p-Akt Ser473 Ab (CST-9271), rabbit anti-JAK1 Ah (sc-7228), rabbit anti-JAK2 Ab (sc-294), rabbit anti-JAK3 Ab (sc-513), rabbit anti-TYK2 Ab (sc-169), rabbit anti-cleaved caspase-3 Asp175 Ab (CST-9661), mouse anti-full-length caspase-3 Ab (sc-7272), mouse anti-caspase-8 Ab detecting both full length and active fragments (CST-9746), rabbit anti-caspase-9 Ab detecting both full length and 35/37 kDa cleaved fragments (CST-9502), rabbit anti-PARP Ab (CST-9542), rabbit anti-BclxL Ab (sc-7195), goat anti-actin Ab (sc-1615), mouse anti-Bax MAb (sc-7480), mouse anti-Bcl-2 MAb (sc-509), goat anti-COX-1 Ab (se-1752), and goat anti-COX-2 Ab (sc-1746). Indomethacin (17378) was from Sigma (St. Louis, Mo.) and the NS-398 (#70590) from Cayman Chemical (Ann Arbor, Mich.).

Antibodies for flow cytometry (FACS) from the Beckman Coulter Company (Miami, Fla.) were: anti-CD1a-PE (IM1942), anti-CD3-PE (IMI282), anti-CD10-PE (IMI915), anti-CD11a-FITC (TM0860), anti-CD11b-PE (IM2581), anti-CD11c-PE (IM1760), anti-CD13-PE (IM1427), anti-CD14-FITC (IM0645), anti-CD15-FITC (IM1423), anti-CD16-FITC (IM0814), anti-CD28-FITC (IM1236), anti-CD33-PC5 (IM2647), anti-CD34-PC5 (IM2648), anti-CD38-FITC (TM0775), anti-CD40-PE (IM1936), anti-CD41-FITC (IM0649), anti-CD45-FITC (IM0782), anti-CD45RA-FITC (IM0584), anti-CD45RO-PE (IM1307), anti-CD49d-FITC (IM1404), anti-C-D49e-FITC (IM1854), anti-CD51-FITC (IM1855), anti-CD54-PE (IM1239), anti-CD61-FITC (IM1758), anti-CD80-FITC (IM1853), anti-CD83-PC5 (IM3240), anti-CD86-PE (IM2729), anti-CD117-PC5 (IM2657), anti-CD152-PE (IM2282), and anti-HLA-DR-FITC (IM1638). The anti-CD68-FITC (GM-4152) was from Caltag Laboratories (Burlingame, Calif.).

Cell cultures—Cultures of foreskin-derived human fibroblasts, HFSF-132, were used from passages 7 to 15 as described (Bizik et al, 2004). KG-1, TET-1, U-937, K562, Jurkat, and Raji were from the American Type Culture Collection (ATCC, Manassas, Va.). All cells were cultured in RPMI 1640 (Life Technologies, Paisley, Scotland) supplemented with 10% fetal bovine serum (Life Technologies), 100 Ag/mL streptomycin, and 100 units/mL penicillin.

Spheroid formation was initiated as described by Bizik et al, 2004. Briefly, U-bottom 96-well plates (Costar, Cambridge, Mass.) were treated with 0.8% LE agarose (BioWhittaker, Rockland, Me.) prepared in sterile water to form a thin film of a nonadhesive surface. Fibroblasts were detached from culture dishes by trypsin/EDTA, and a single cell suspension (4×10⁴ cells/ml) was prepared in a complete culture medium. To initiate spheroid formation, 250 ml aliquots were seeded into individual wells and the dishes incubated at +37° C. in a 5% CO₂ atmosphere.

For the co-culture and nemosis stimulation experiments, the leukemia cells were cultured for various time-periods with 24-hour-preformed fibroblast spheroids at a 1:1 leukemia cells:fibroblast ratio. For the estimation of growth curves, cell numbers were evaluated by cell-counting in Bürker chambers. For immunoblotting, FACS, and adherence testing, the residual spheroids were removed from co-cultures by gravitational differential sedimentation.

Morphology of leukemic cells 96 hours after co-culturing was evaluated by phase contrast microscopy. The leukemic cells' adherence was estimated after 96 hours of co-culturing with fibroblast spheroids. Thereafter aliquots of cell lines were seeded onto standard cell-culture dishes for 24 hours. The cultures were washed, and adherent cells were harvested by trypsinization, were counted, and the percentage of these adherent cells was calculated.

Chemotaxis of leukemic cells was performed in agarose-treated 6-well plates as co-cultures of 24-hour-preformed fibroblast spheroids with the naive leukemia cell lines. We calculated with an ocular grid the number of leukemic cells located at a distance from the spheroid double its own diameter, and measured these cells around 15 spheroids per well.

Cell cycle analysis—For DNA histograms and cell cycle analyses, leukemic cells were co-cultured for 96 hours with 24-hour-preformed fibroblast spheroids, and separated from fibroblast clusters by sedimentation. These cells were then washed with PBS and fixed in 1% paraformaldehyde, were treated with RNAse (100 μg/ml), their DNA was stained with propidium iodine (50 μg/ml), and they were analyzed by FACS.

Immunoblotting—Cell samples were lysed directly in SDS-PAGE sample-loading buffer: 62.5 mmol/L Tris-HCl (pH 6.8), 2% SDS, 20% glycerol, 5% β-mercaptoethanol, and 0.005% bromophenol blue, supplemented with Complete Mini-protease inhibitor mixture tablets (Roche, Mannheim, Germany) and boiled for 5 minutes. Lysates were centrifuged at 14,000 rpm for 15 minutes to sediment particulate-insoluble material. These samples were separated in SDS-PAGE (gradient of polyacrylamide 5-15%, 3.5% stacking gel). The proteins were transferred electrophoretically from the gel to a nitrocellulose membrane (Schleicher & Schuell, Dassel, Germany), with transfer efficiency verified by Ponceau-S staining. After blocking of the membrane with 2.5% low-fat dry milk in TBS, 20 mmol/L Tris-HCl, 150 mmol/L NaCl, and 0.1% Tween 20 at pH 7.5′ it was incubated with specific primary antibodies, followed by an alkaline phosphatase-conjugated secondary antibody (Promega, Madison, Wis.). Protein bands were visualized according to manufacturer's recommendations.

Flow cytometry—For flow cytometric analysis, the leukemia cells co-cultured for indicated time points and after differential sedimentation to remove spheroids were incubated on ice with antigen-specific antibodies or with isotype-matched antibodies as controls, and fixed in 1% paraformaldehyde. FACS analysis was done by an EPICS ALTRA flow cytometer with the EXPO32 analysis program (both from Beckman Coulter Inc, Fullerton, Calif.).

Measurements of cytokine concentrations by enzyme-linked immunoassays—Fibroblast spheroid-conditioned medium was collected at 96 hours after initiation of spheroid formation from the 96-well plates. Concentrations of IL-1β, IL-6, IL-8, IL-11, GM-CSF, LIF, oncostatin M, and TNF-α were quantified by commercial ELISA kits and reagents according to manufacturers' instructions. The human IL-1β, human IL-11, human LIF, human TNF-α, and human oncostatin M ELISAs were from R&D Systems (Minneapolis, Minn.), the human IL-6 and human IL-8 ELISAs were from the Central Laboratory of the Netherlands Red Cross (CLB, Amsterdam). Cytokine quantification in the nemotic fibroblast-conditioned medium for human IL-2, IL-4, IL-5, IL-10, IL-12, IL-13, GM-CSF, interferon-γ (IFN-γ), and TNF-α was carried out with the Bio-Plex Human Cytokine Th1/Th2 Panel (Bio-Rad Laboratories Inc, Hercules, Calif., catalogue number 171-A11081), by the Luminex 100 System (Luminex Corporation, Austin, Tex.).

### Results

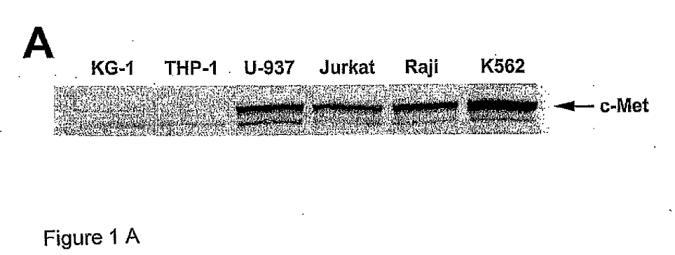

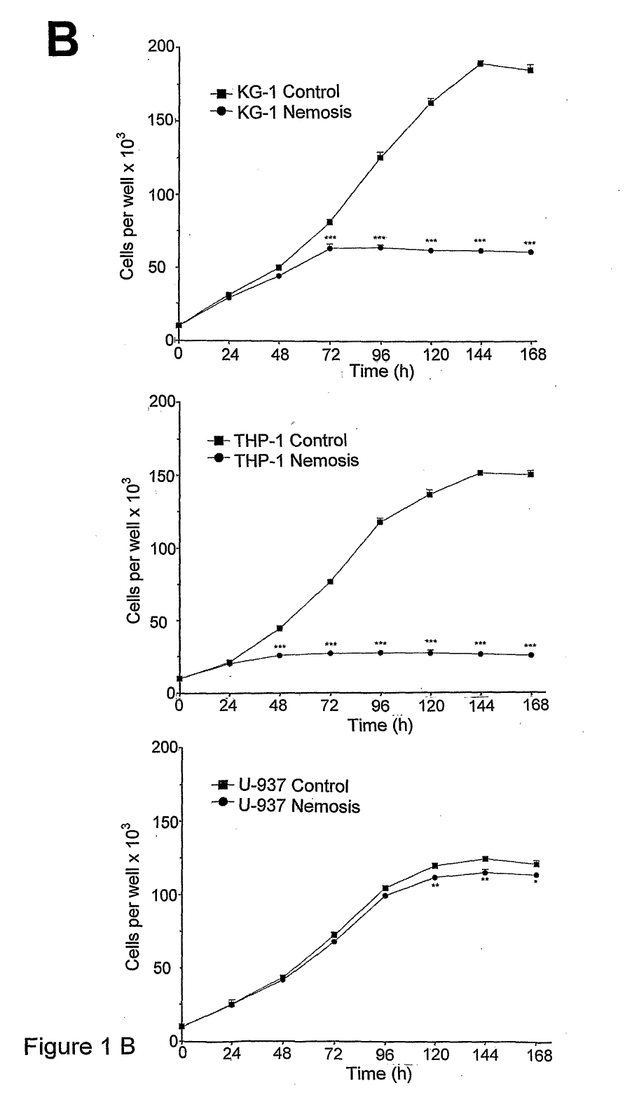

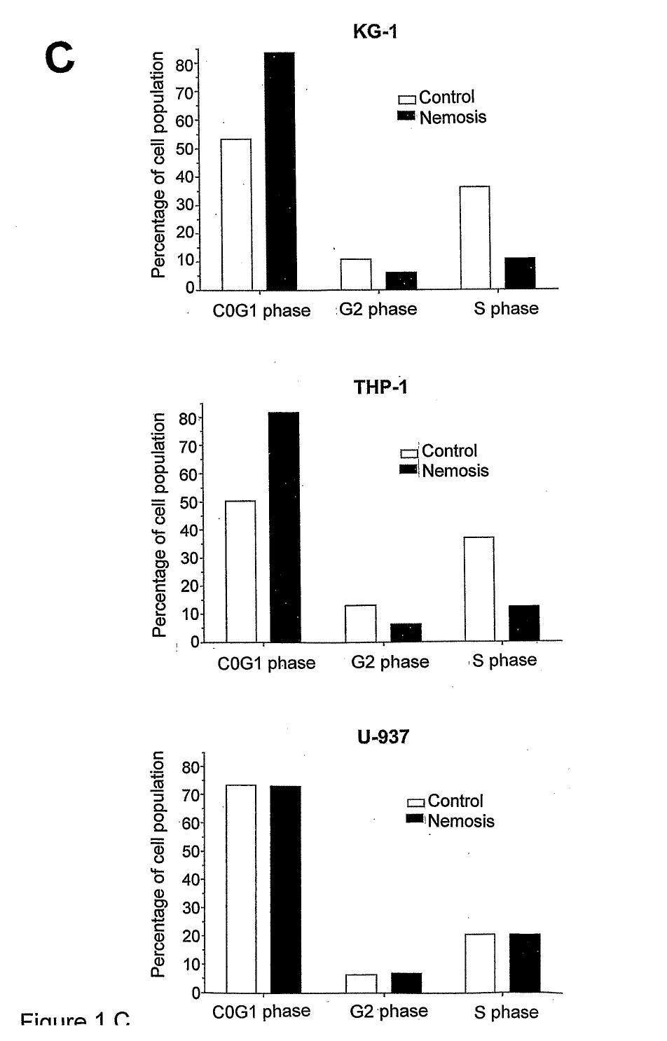

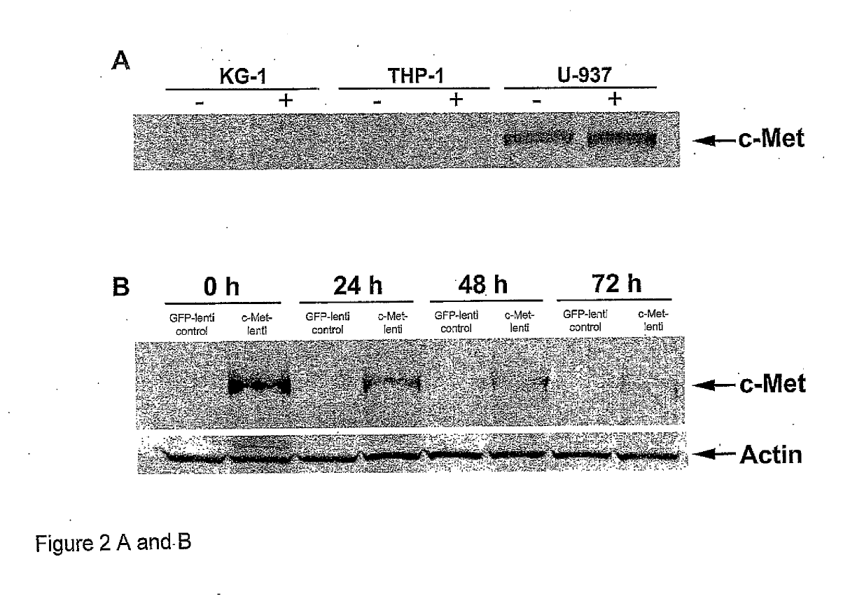

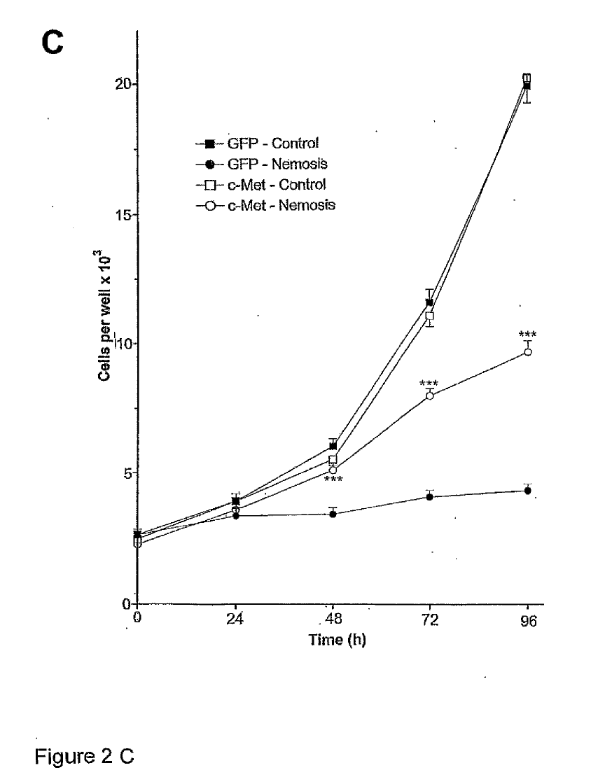

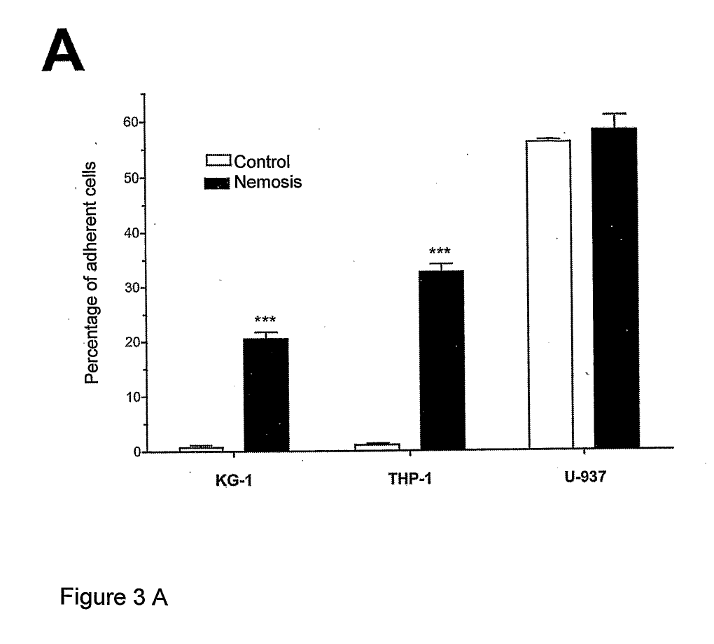

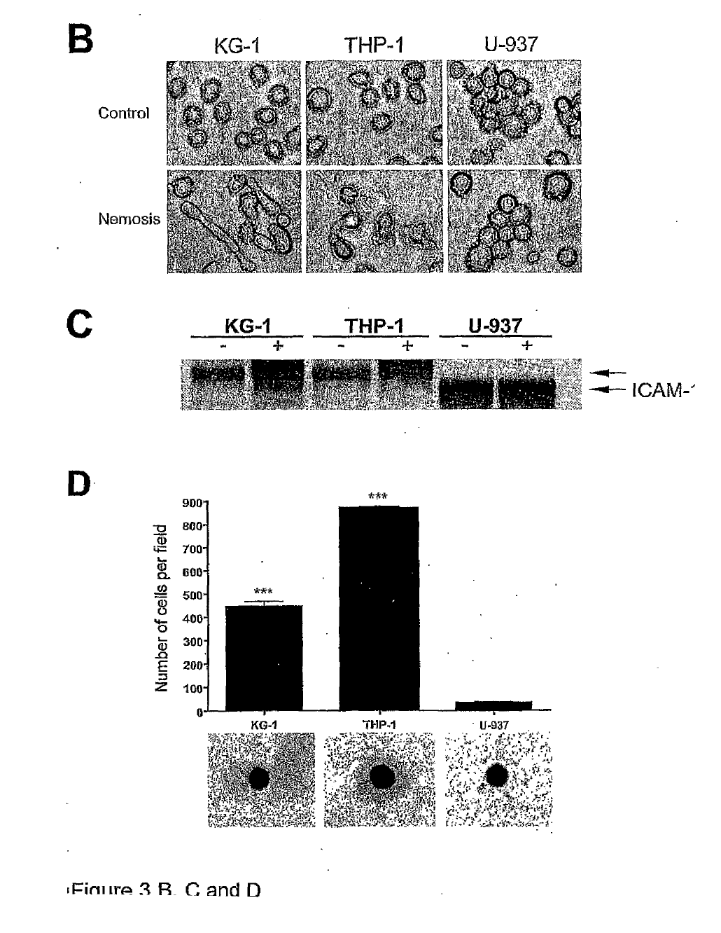

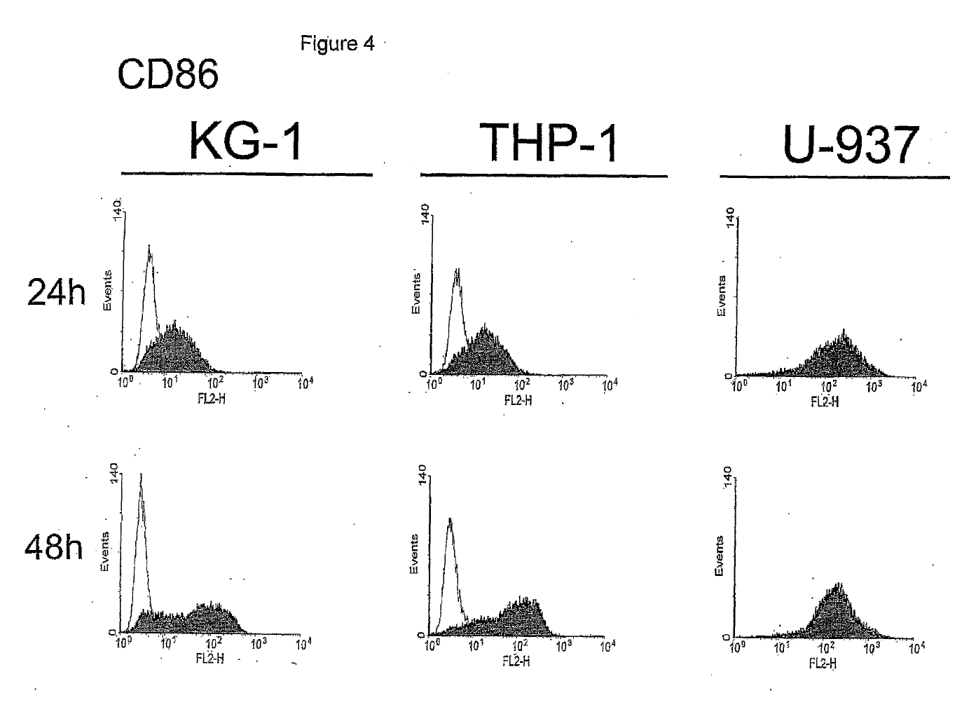

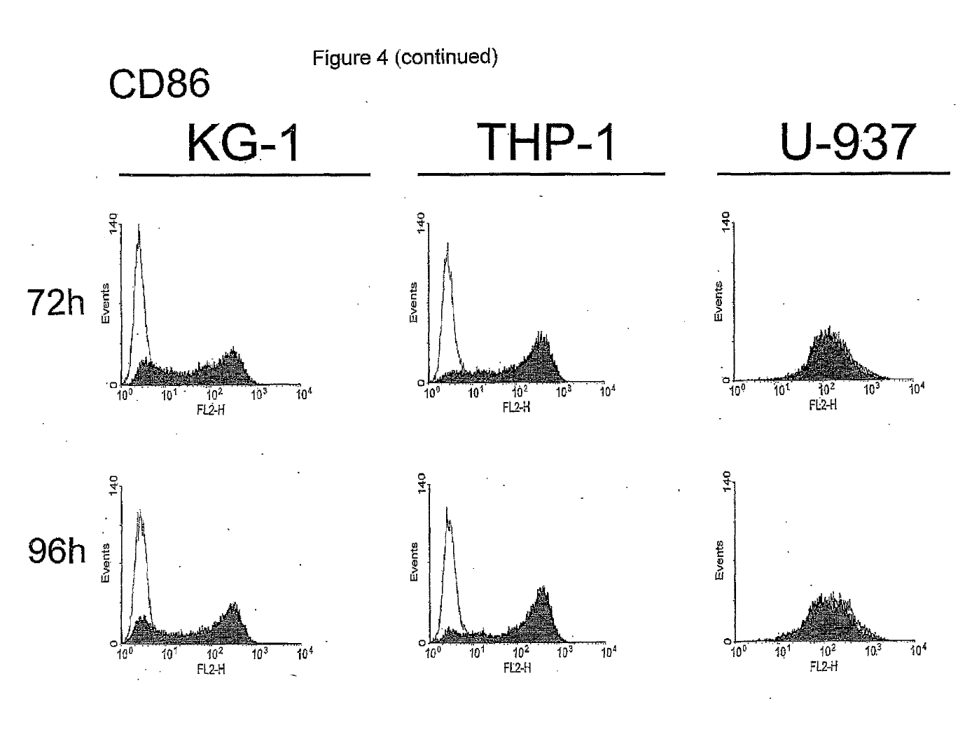

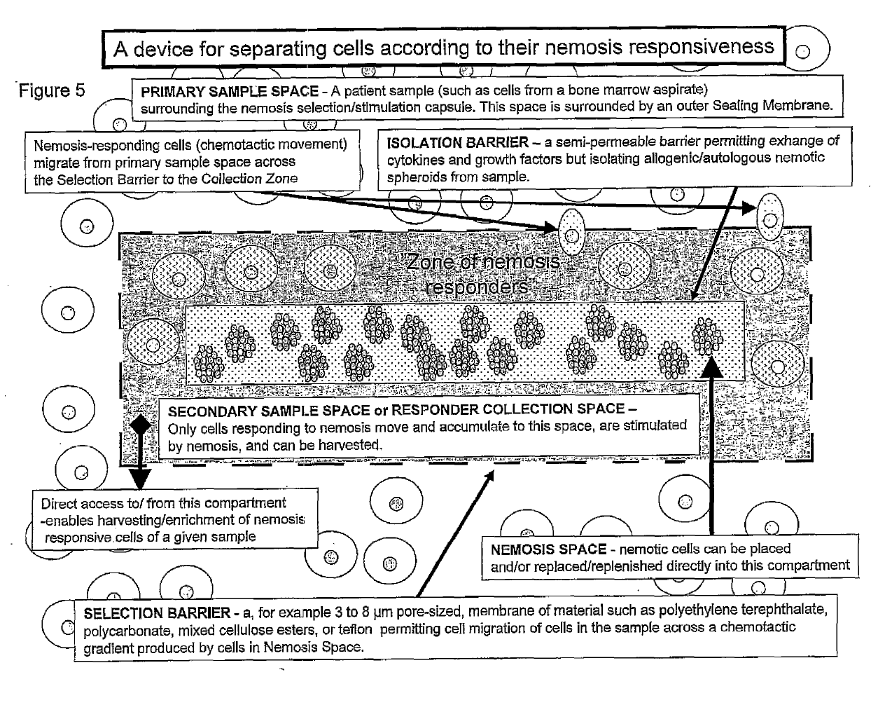

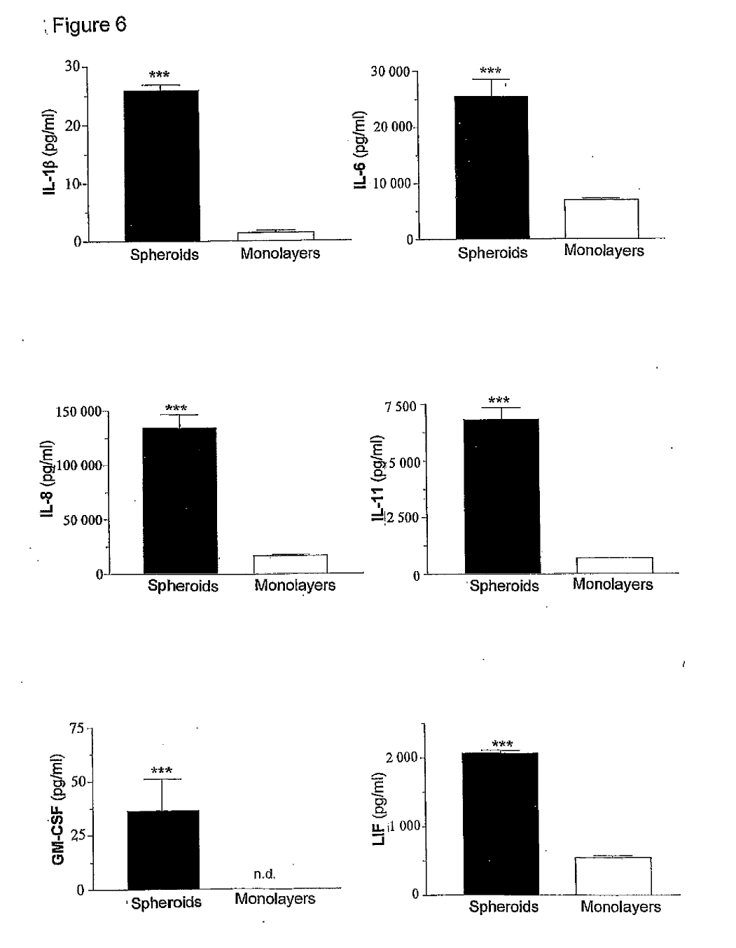

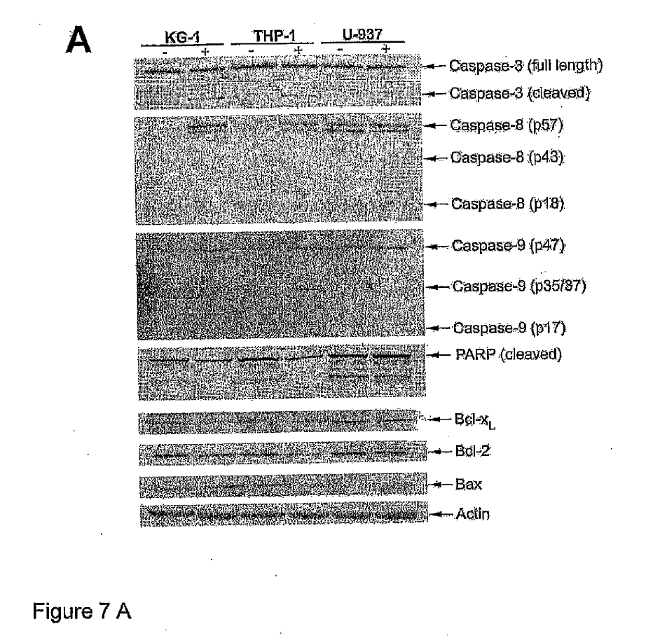

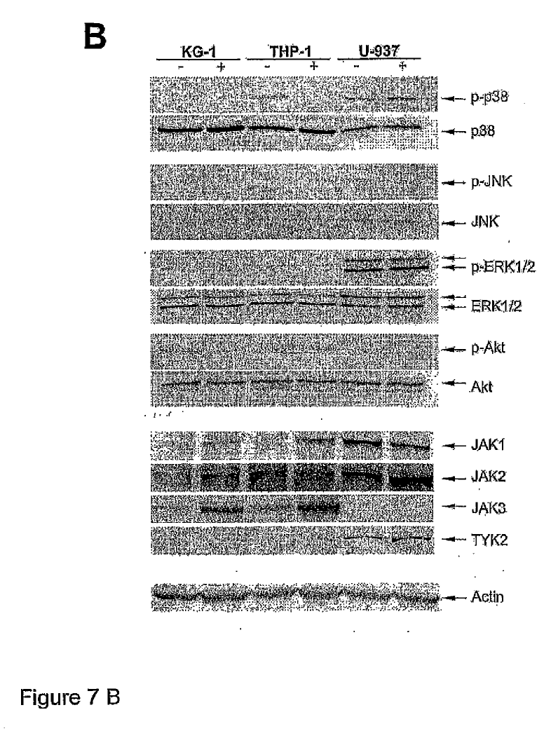

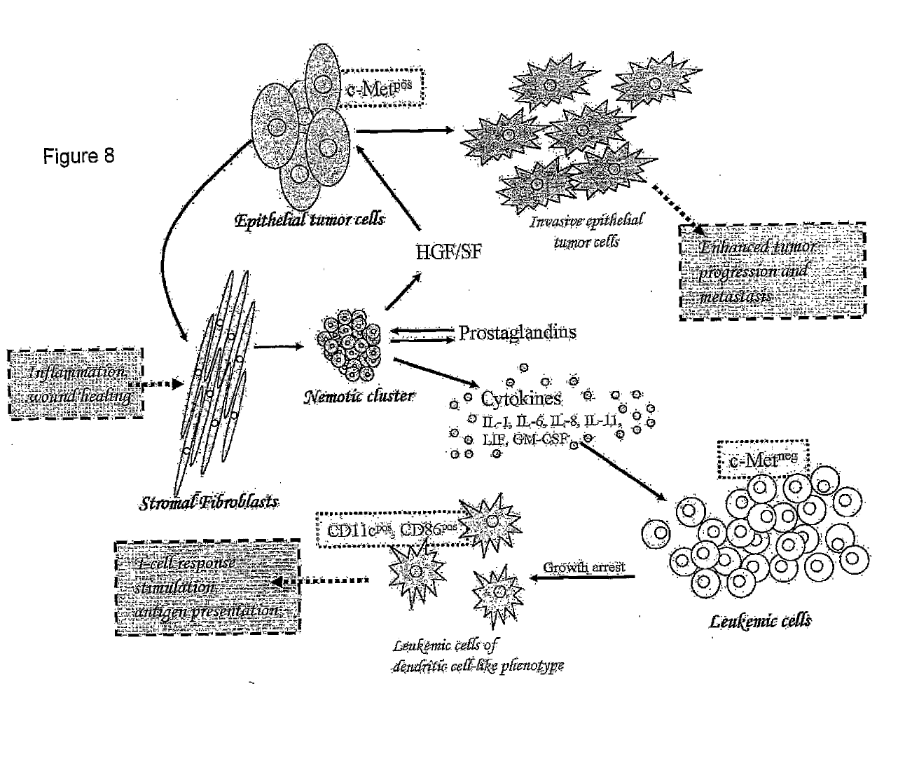

Phenotypic and growth characteristics and cell cycle analysis—In analysis of leukemia cell lines for their expression of the HGF/SF receptor c-Met, expression of the properly processed form appeared on U-937, Jurkat, Raji, and K562 cell lines blut not on THP-1 or KG-1 cells (FIG. 1A). We screened all these cells in co-culture with nemotic fibroblasts for their growth characteristics. The c-Met-positive cell lines showed no significant alterations in their proliferation rates, but the c-Met-negative cell lines THP-1 and KG-1 responded with discernible growth arrest. For subsequent experiments on stimulation with nemotic fibroblasts, we chose the c-Met-negative THP-1 and KG-1 cell lines, and the c-Met-positive U-937 cell line. Stimulation of these cells by nemesis produced no effect on their expression levels of c-Met (FIG. 1B). The THP-1 and KG-1 vs. the U-937 cells also differed in their expression of focal adhesion kinase (FAK) and cyclooxygenase-1 (COX-1) (FIG. 1B). The inducible COX-2-isoform in these cell lines was, however, undetectable (data not shown).

Cell cycle analysis of KG-1, THP-1, and U-937 cells—In co-culture, nemotic fibroblasts induced a dramatic growth inhibition of the cells lacking c-Met, whereas growth of the c-Met-positive cell lines showed only marginal, if any, inhibition. FIGS. 2A to 2C show the growth responses when these cell lines were subjected to fibroblast nemosis. Attenuation of growth by nemosis treatment was evident at 72 hours of co-incubation for the KG-1 cells, but was already evident at 48 hours in the THP-1 cell line, demonstrating the more rapid response and reactivity to nemosis of the latter cells. After 96 hours of incubation, only a modest and delayed effect on proliferation was apparent in U-937 cells. The nemosis-arrested proliferation of the responder cell lines persisted throughout the study, with the control population reaching a growth plateau after an exponential growth phase. With the control cells reaching their growth plateau at 168 hours, nemosis inhibited the proliferation of the cell lines by 67% for KG-1, 83% for TBT-1, and 6% for U-937 cells.

FIGS. 2D to 2E show the leukemic cell lines' cell-cycle phase distribution as evaluated by DNA histograms. When treated with nemesis, 30.2% of the KG-1 and 31.3% of the THP-1 cell populations accumulated in the G0G1 phase. This shift away from the M and S phases indicates cell-cycle arrest associated with differentiation. The U-937 cells retained their cell-cycle characteristics, with no apparent population shift in response to fibroblast nemosis. Moreover, treatment of clustering fibroblasts with the non-steroidal anti-inflammatory drugs (NSAIDs) NS-398 and indomethacin to prevent prostaglandin production (Bizik et al, 2004) had no effect on inhibition of proliferation by nemosis (data not shown).

Morphological and functional characteristics of leukemic cells in response to nemosis—Because arrest of the cell cycle at the G0G1 phase is associated with induction of differentiation (Liu et al, 1996), we evaluated changes in morphological characteristics and adherence of the leukemia cells in response to nemosis. Once more, the differences between nemosis-responders KG-1 and THP-1, and the U-937 control unresponsive cell line were distinct. In KG-1 and THP-1 cells, nemosis led to an increased proportion of adherent cells by 19.8 and 31.6% (FIG. 3A). These cells showed morphological features of a dendritic-cell-like phenotype with cell elongation and formation of stellate pseudopodia (FIG. 3B), but in response to nemosis, the U-937 cells changed neither their morphology nor their pattern of adherence (FIG. 3B). Induction of an adherent phenotype in the nemosis-responsive cell lines was associated with increased expression of intercellular adhesion molecule-1 (ICAM-1) (FIG. 3C).

Increased adherence of KG-1 and THF-1 cells by nemosis suggests that the clustered fibroblasts can also produce factors affecting cell motility and chemotaxis. We therefore evaluated the chemotactic responses of KG-1, THP-1, and U-937 cells in co-culture with the nemotic fibroblasts. Comparison of the responses of the analyzed cell lines showed that both KG-1 and THP-1 were chemotactically drawn towards the fibroblast clusters undergoing nemosis, whereas the U-937 cells were unresponsive (FIG. 3D). Compared to the U-937 cells, nemosis attracted the KG-1 and THP-1 cells to accumulate in the vicinity of the fibroblast clusters at an 11- and a 22-fold enhanced density.

Since monocyte maturation and differentiation are associated with decreased antigen uptake through macropinocytosis (Austyn, 1996), we studied, as a parameter of differentiation on a functional scale, the effect of nemosis on pinocytotic activity. The ability of the leukemia cell lines to repel FITC-labeled dextran was assessed at 24 and 96 hours of co-incubation of leukemia cells with nemotic spheroids. After stimulation of the cell lines with nemosis followed by incubation with FITC-dextran, flow cytometry revealed a distinct inhibition of FITC-dextran uptake in nemosis-treated KG-1 (mean intensity change −11.92) and THP-1 cells (mean intensity change −3.86), with the U397 cells showing an increase of 0.91 in intensity (data not shown). These data are in accordance with morphological characterization showing induction of adherence and the presence of dendritic cell-like pseudopodia on the nemosis-responsive KG-1 and THP-1 cells.

Changes in surface antigen expression of nemosis-stimulated cell lines—For further phenotypic characterization of nemosis-treated cells, we carried out FACS analysis of the cell-surface antigens, as shown in the Supplementary Table, by comparison of control antigen intensity to that of nemosis-treated cells. From this analysis, a clear induction pattern of five cell-surface markers emerged. Interestingly, and in accordance with the other cell responses, these antigens were induced only in KG-1 and THP-1 cells. No induction of these surface antigens was evident in U-937 cells, which responded with a slight overall expressional down-regulation.

By this approach, we identified in the nemosis-responsive cell lines induction of the following: dendritic cell marker CD11c, the leukocyte common antigen CD45RA, the adhesion molecule CD54, the dendritic cell-associated T-cell co-stimulatory molecule CD86, and the membrane peptidase CD13. The time-dependence of CD86 induction (FIG. 4) showed response kinetics similar to the growth arrest in KG-1 and THP-1 cells, with the latter cell line reacting more promptly also by this parameter. No effect on the induction of CD86 occurred when, prior to co-culture with the leukemia cells, fibroblast clusters were formed in the presence of NS-398 or indomethacin (data not shown).

Further population analysis was carried out based on differential expression of CD45 in various lineages and differentiation stages (Stelzer et al, 1993). By gating on CD45 we identified a new subpopulation with high granularity (as defined by high scatter-count values) in nemosis-treated KG-1 and THP-1 cells, but no changes occurred in response to nemosis in the subpopulation characteristics of U-937 cells. The emerging populations were positive for all the nemosis-induced markers, especially for CD11c and CD13, in contrast to a non-responsive similar CD45-positive population of U-937 cells. Nemosis caused increased CD86 positivity in the CD45-positive population of KG-1 and THP-1 cells with low SSC values. Considered together, these results suggest that in responsive cell lines nemosis induces expression of antigens involved in antigen presentation and T-cell stimulation, along with dendritic cell characteristics.

Cytokine production in nemotic fibroblasts—We previously reported that nemotic fibroblasts are a rich source of the c-Met ligand HOF/SF. Based on our data, and the lack of any effect by NSAIDs, it seemed obvious that the lack of c-Met in the nemosis-responsive leukemia cell lines ruled out any role for HOF/SF in induction of growth arrest. Moreover, the chemotactic response suggests involvement of chemoattractants. We therefore evaluated a pattern of cytokines known to be associated with modulation of chemotaxis and leukemia cell proliferation. Distinct induction of interleukin(IL)-1β, IL-6, IL-8, IL-11, granulocyte-macrophage colony-stimulating factor (GM-CSF), and leukemia inhibitory factor (LIF) was evident in nemotic spheroids compared with their release from the corresponding monolayer cultures at 96 hours from culture initiation (FIG. 6). The levels of IL-2, IL-4, IL-5, IL-1, IL-12, IL-13, interferon-γ, oncostatin M, and TNF-α remained low or undetectable and remained unaffected by cell culture arrangement. The cytokines produced most abundantly by the spheroids were IL-6 and IL-8, with fold-inductions (mean production in spheroids) of 3.7 (25.4 ng/ml) and of 8.0 (158.7 ng/ml) as compared to the corresponding monolayer cultures. In the fibroblast spheroids fold-inductions of 18.3 and 3.8 occurred in the production of IL-1β and LIF. Nemotic fibroblasts also produced GM-CSF, which was undetectable from monolayer cultures (FIG. 6). These results suggest that nemosis induced by fibroblast clustering is a fundamental source of an array of cytokines and growth factors regulating monocyte functions.

Apoptosis-related intracellular changes in the leukemia cell lines by nemosis—Inhibition of tumor cell growth is usually accompanied by induction of apoptosis. We therefore evaluated the known apoptotic pathways in the growth-arrest and differentiation responses of THP-1 and KG-1 cells to nemosis. In FIG. 7A, expression of several apoptosis-associated proteins revealed that the cleaved, active form of the universal apoptosis executor, caspase-3, occurs in response to nemosis only in the nemosis-responsive cell lines. That the unresponsive U-937 showed no effect suggests activation of apoptosis in the nemosis-responsive cells. No changes in the expression of the active cleaved forms of the initiator caspases-8 and -9 were detectable in any of the cell lines, but expression of the full-length caspase-8 was induced by nemosis in the THP-1 and KG-1 cells. In this case, as well, the U-937 cells showed no effect. The increases in caspase-3 cleavage and caspase-8 expression were not, however, reflected in the expression of the apoptosis-regulating Bcl-xL, Bcl-2, or Bax proteins, suggesting their apoptosis-un-elated mechanism of action.

Due to the evident cleavage and increased expression of the active form of caspase-3 caused by nemosis in THP-1 and KG-1 cells, we evaluated the extent of poly-ADP-ribose-polymerase (PARP) cleavage associated with DNA damage, apoptosis, and caspase-3 activity (Ame et al, 2004). FIG. 7A shows the expression pattern of full-length PARP (p116) and its cleaved inactive form p89 in leukemia cell lines subjected to nemosis. That nemosis treatment inhibited PARP cleavage in THP-1 and KG-1 cells, with the U-937 cells left unresponsive, suggests that in these cell lines, increased caspase-3 cleavage is unrelated to apoptosis induction in terms of PARP inactivation.

Effects of nemosis oil kinase cascades in the leukemic cell lines—Dysregulation of the mitogen-activated protein kinase (MAPK) pathways has been associated with the highly proliferative phenotype of leukemia cells (Platanias, 2003). We evaluated the involvement of MAPKs c-jun N-terminal kinase (JNK), extracellular signal-regulated kinases p44/p42 (ERK1/2) and p38 in leukemia cell responsiveness to nemosis (FIG. 7B). In all the naive leukemia cell lines, we found constitutive phosphorylation of these kinases, reflecting their mitotically active phenotype. In the KG-1 and THP-1 cell lines, which responded to nemosis with growth arrest and differentiation, phosphorylation of p38 MAPK was dramatically quenched, whereas no changes in p38 MAPK phosphorylation in response to nemosis occurred in the U-937 cell line. A similar but less pronounced inhibition of phosphorylation was evident in ERK1/2, with the U-937 cells again left unresponsive to nemosis. The level of phosphorylation and expression of JNK in all cell lines remained relatively unchanged. The high nemosis-unresponsive level of phosphorylation of p38 MAPK, JNK, and ERK1/2 in U-937 cells, as compared to that in cell lines TIP-1 and KG-1, with a sustained nemosis-unaffected phosphorylation of Akt suggests that these phenotypically different leukemic cells utilize different pathways for high proliferative activity. It also suggests that in nemosis-responsive cells, the downregulation of p38 and ERK1/2 MAPKs is associated with growth arrest and differentiation.

We also evaluated the expression of the Janus protein tyrosine kinase family (JAK) members (JAK1, JAK2, JAY3, and TYK2) known to be associated with monocyte and leukemia cell differentiation (Rane et al, 2002; Mangan et al, 2004). In the KG-1 and THP-1 cells induced to undergo differentiation by fibroblast nemosis, expression of JAK1 and JAK3 was increased. In the non-responsive U-937 cell line, expression of JAK1 was downregulated by nemosis, with no visible expression of JAK3 (FIG. 7B). Phenotypic differences between U-937 and the nemosis-responsive KG-1 and THP-1 cells were reflected in TYK2 as well as in JAK3 expression. JAK2 expression correlated neither with growth arrest nor with differentiation (FIG. 7B).

### Discussion

We present data showing that clustered fibroblasts are able to induce a cytostatic, growth inhibitory, and differentiating response in the c-Met-negative leukemia cell lines THP-1 and KG-1, whereas no effect was observed on the c-Met-positive U-937 cell line. We also show that nemotic fibroblasts are rich producers of cytokines and growth factors such as IL-1β, IL-6, IL-8, IL-11, LIF, and GM-CSF, all of which are involved in regulation of hematopoiesis and differentiation (Lotem & Sachs, 2002; Zhu & Emerson, 2002). These results are in agreement with our previous data (Bizik et al, 2004; Kankuri et al, 2005) and suggest that nemotic fibroblasts can produce high amounts of paracrine mediators with their effect largely determined by the phenotype and receptor-expression profile of the target cells.

Tumor cells, showing an imbalance between cell survival and death, have adopted an immature or undifferentiated phenotype (Bishop, 1991; Wang & Chen, 2000). Their persistence is further promoted by their ability to evade recognition by the adaptive immune system (Zou, 2005), Leukemic cells represent an undifferentiated phenotype of white blood cells, and inducing their differentiation toward a dendritic cell-like type has stimulated therapeutic antileukemic T-cell responses (Charbonnier et al, 1999; Choudhury et al, 1999; Cignetti et al, 1999; Fujii et al, 1999; Claxton et al, 2001; Mohty et al, 2002; Cignetti et al, 2004). Differentiation of neoplastic cells can thus show therapeutic benefit in hematopoietic malignancies (Reiss et al, 1986).

In addition to induction of growth arrest, we show that nemotic fibroblasts can also drive a phenotype-dependent differentiation of leukemia cells in terms of morphological features, functional responses, and surface antigen expression. Differentiation of THP-1 and KG-1 cells in response to nemosis clearly represents an adherent, mature antigen-presenting cell-like type, as further characterized by FACS. For flow-cytometric evaluation and cell population-based characterizations, we used CD45-gating. CD45 and CD45RA are expressed on all hematopoietic cells except mature red blood cells and their immediate progenitors, with increased levels of these antigens correlating with degree of differentiation (Hermiston et al, 2003). Consequently, their expression is also useful in clinical determination of lymphoid and myeloid cell maturity (van Lochem, 2004). Using FACS-gating on CD45, we found an emerging population with high granularity based on size-scatter values, and with enhanced expression of CD11c, CD86, CD54, and CD13 cell-surface antigens in the nemosis-responder cell lines. When subjected to nemotic fibroblasts, increases in the specific dendritic cell marker CD11C and the T-cell co-stimulatory CD86 were evident also in the population with low SSC values of these cells.

Increased expression of the β2 integrin CD11c, a marker for myeloid dendritic cells (Osugi et al, 2002) associated with differentiation and maturation (Corbi & Lopez-Rodriguez, 1997; Noti & Reinemann, 1995), we found to be linked to the induction of co-stimulatory molecule CD86 in the nemosis-responders. CD11c acts as an adhesion molecule mediating cell-cell and cell-matrix interactions (Shelley et al, 2002). On the other hand CD86 (B7-2) binds CD28 and CTLA-4 molecules on T-cells mediating co-stimulatory signaling (Collins et al, 2002) to enhance T-cell proliferation, activation, and clonal expansion (Coyle et al, 2001). Stimulation of T-cell CTLA-4 enhances antitumor activity and of CD28 enhances the cytotoxic T-lymphocyte-mediated destruction of tumors (Zheng et al, 1998). Similar to CD11e, expression of CD86 on leukemia cells has been associated with a dendritic cell-like phenotype (Re et al, 2002).

Our work shows that the cell lines KG-1 and THP-1 attained a more adherent phenotype in response to fibroblast nemosis. Based on our FACS analyses, this is explained not only by induction of CD11c in these cells but also by increased positivity for CD54 (ICAM-1), which is expressed in both hematopoietic and non-hematopoietic cells mediating adhesive interactions, for example by binding to the β2 subfamily integrins CD11a/CD18 (LFA-1) and CD11b/CD18 (Mac-1) (Marlin & Springer, 1987; Roebuck & Finnegan, 1999). Similar to CD11c, ICAM-1 also mediates transendothelial migration of leukocytes and, like CD86, ICAM-1 binding functions as a co-stimulatory signal for the activation of T cells in antigen presentation (Zuckerman et al, 1998; Grakoui et al, 1999; Hubbard & Rothlein, 2000).

By phenotyping, we discovered evidence that nemosis treatment induces dendritic cell-like expression of surface antigens on leukemia cell lines in a target-cell phenotype-dependent manner. Moreover, our results also suggest that the phenotype acquired via nemosis is reminiscent of mature antigen-presenting cells with lower activity for macropinocytosis. The association with antigen-presenting cells was further strengthened by our observation of increased CD13, an aminopeptidase which cleaves peptides for presentation on major histocompatibility complex class II molecules and participates in antigen processing (Larsen et al, 1996).

In order to extend our data to the molecular intracellular level, we investigated the involvement of two major pathways associated with growth arrest and differentiation: activation of key players in the apoptosis cascade and phosphorylation of some MAP kinase signaling pathways. What we found was intensive cleavage of the executor caspase-3, in contrast to unchanged expression of Bcl-2, Bcl-2X_{L}, and Bax, accompanied by no changes in cleavage of the initiator caspases-8 and -9. In contrast to the executor caspases requiring protecolytic cleavage for activation, the initiators are also activated without cleavage by oligomerization or dimerization (Salvesen & Abrams, 2004). Therefore, the increased level of uncleaved full-length caspase-8 with no signs of cleavage does not rule out its activation in the nemosis-responsive cell lines THP-1 and KG-1. Surprisingly, despite the evident association between apoptosis and caspase-3 activation by cleavage, no increased proteolytic processing of PARP, the caspase-3 substrate associated with DNA-repair, occurred; this suggests for caspases-3 and -8 an apoptosis-unrelated differentiating function.

PARP has several functions, and its increased activity and expression are associated with damage to DNA and DNA repair, with cell proliferation and differentiation, and with regulation of transcription (Ame et al, 2004). The role of PARP as a causal effector of apoptosis is equivocal (Leist et al, 1997). We never found increases in protein levels of Bax, Bcl-2, and Bcl-2X_{L} to be associated with reduced PARP expression, suggesting an apoptosis-unrelated mechanism for PARP downregulation. Moreover, when cell differentiation progresses, the activity and expression of PARP decreases (D'Amours et al, 1999; Virag & Szabo, 2002), suggesting that the decreased PARP expression and cleavage we saw reflect cellular differentiation and metabolic inhibition as well as growth arrest. For example, PARP was dramatically downregulated in response to all-trans-retinoid acid-induced NB4 cell neutrophilic differentiation (Bhatia et al, 1995), and this effect was blocked by PARP overexpression (Bhatia et al., 1996). Despite a possible causal effect on differentiation, our results lead to speculation that PARP expression and activity reflect the high mitotic rate of leukemic cells; after they undergo growth arrest they have less need for PART expression and activity in the nucleus to protect the fragile opened DNA of mitosis (D'Amours et al, 1999).

Caspases 3 and 8 are important regulators of differentiation processes not only in monocytes but also in skeletal muscle cells and osteoblasts (Launay et al, 2005). In hematologic and other types of malignant cells, downregulation of caspase-8 serves as a means to resist apoptosis (Hopkins-Donaldson et al, 2003, Yang et al, 2003). Upregulation of caspase-8, as shown here by the action of fibroblast nemosis on leukemic cells, thus suggests that these nemosis-responsive cells act in an opposite-and in a more benign-manner. Furthermore, the fact that we found no decrease in cell number after nemosis treatment but rather a dramatic cessation of mitosis strengthens the association between growth arrest and the strikingly similar phenotype of both cell lines. This implies that in the nemosis-responsive phenotype, differentiation is favored over apoptotic death.

Alterations in MAPK cascades involving JNK, ERKs, and p38s have been associated with malignant cell proliferation, differentiation, and death (Platanias, 2003). Of our cell lines tested, none responded to nemosis with changes in JNK expression or phosphorylation. However, the phosphorylation of p38 MAPK—and to a minor extent also that of ERK1/2—was significantly suppressed in those cell lines responding to nemosis with differentiation and growth arrest: namely the THP-1 and KG-1 cells. The literature reveals that the role of p38 MAPK in cell death and differentiation is enigmatic (Platanias, 2003), and that its activity in normal and malignant cells may be differentially regulated (Engelberg, 2004). A growing pool of data suggests, however, that active p38 MAPK acts as an enhancer of cell survival (Villunger et al, 2000). Moreover, p38 has been shown to prevent Jurkat T-cell apoptosis (Nemoto et at, 1998), and to inhibit all-trans-retinoid acid-induced differentiation of one acute pro-myetocytic cell line (Alsayed et al, 2001).

This is in agreement with our data showing that inhibition of the p38 MAPK phosphorylation reflecting kinase activity occurs only in those cells responding to nemosis with differentation and growth arrest. It thus suggests that the highly proliferating phenotype is associated with active p38 MAPK and may indicate an altered or constitutively active p38 pathway in these cells. Moreover, when the responsive cell lines are subjected to fibroblast nemosis, induction of the JAK kinases JAK1 and JAK3 occurs. This is concordant with the prevailing view of expressional control of 1AK3 (Rane & Reddy, 1994; Mangan et al, 2004) and to some extent also of JAK1 (Rodig et al, 1998) in hematopoietic regulation and differentiation. Induction of the JAKs in the nemosis-responsive cell lines suggests involvement of IL-6 and GM-CSF in mediating the effect of differentiation.

As induced by tumor cell-derived factors (Kankuri et al, 2005), cell clustering-induced nemotic signaling of fibroblasts presents a unique type of inherent stromal activation capacity. Through direct effects on differentiation of tumor cells of hematological origin, nemosis may influence responses of the immune system to malignancy (FIG. 8). Differentiation of leukemic cells into the dendritic cell lineage can stimulate anti-leukemic actions of T-cells (Charbonnier et al, 1999; Choudhury et al, 1999; Cignetti et al, 2004); such differentiation can be suggested as immunotherapy (Claxton et al, 2001; Mohty et al, 2002; Buehler et al, 2003). Our results present the first in vitro evidence that homotypic stromal cell-cell interactions leading to nemosis can provide sufficient signaling to modulate and restrain neoplastic growth.

### REFERENCES

Alsayed, Y., Uddin, S., Mahmud, N., Lekmine, F., Kalvakolanu, D. V., Minucci, S., Bokoch, G., and Platanias, L. C. (2001). Activation of Rac1 and the p38 mitogen-activated protein kinase pathway in response to all-trans-retinoic acid, J. Biol. Chem. 276, 4012-4019.
Ame, J. C., Spenlehauer, C., and de Murcia, G. (2004), The PARP superfamily. Bioessays 26, 882-893.
Austyn, J. M. (1996). New insights into the mobilization and phagocytic activity of dendritic cells. J. Exp. Med. 183, 1287-1292.
Babusikova, O., Zeleznikova, T., Mlcakova, A., Kusenda, J., and Stevolova, L. (2005). The knowledge on the 3rd type hematogones could contribute to more precise detection of small numbers of precursor B-acute lymphoblastic leukemia. Neoplasma 52, 502-509.
Bendall, L. J., Daniel, A., Kortlepel, K., and Gottlieb, D. J. (1994). Bone marrow adherent layers inhibit apoptosis of acute myeloid leukemia cells. Exp. Hematol. 22, 1252-1260.
Bhatia, M., Kirkland, J. B., and Meckling-Gill, K. A. (1995). Modulation of poly(ADP-ribose) polymerase during neutrophilic and monocytic differentiation of promyelocytic (NB4) and myelocytic (HL-60) leukaemia cells. Biochem. J. 308, 131-137.
Bhatia, M., Kirkland, J. B., and Meckling-Gilt, K. A. (1996). Overexpression of poly(ADP-ribose) polymerase promotes cell cycle arrest and inhibits neutrophilic differentiation of NB4 acute promyelocytic leukemia cells. Cell Growth Differ. 7, 91 -100.
Bhowmick, N. A., Neilson, E. G., and Moses, H. L. (2004). Stromal fibroblasts in cancer initiation and progression. Nature 432, 332-337.
Bishop, J. M. (1991). Molecular themes in oncogenesis. Cell 64, 235-248.
Bizik, J., Kankuri, E., Ristimäki, A., Taieb, A., Vapaatalo, H., Lubitz, W., and Vaheri, A. (2004). Cell-cell contacts trigger programmned necrosis and induce cyclooxygenase-2 expression. Cell Death Differ. 11, 183-195.
Bradstock, K., Bianchi, A., Makrynikola, V., Filshie, R., and Gottlieb, D. (1996). Long-term survival and proliferation of precursor-B acute lymphoblastic leukemia cells on human bone marrow stroma. Leukemia 10, 813-820.
Bucher, T., Michalek, J., Kovarova, L., Musilova, R., and Hajek, R. (2003) Dendrinic cell-based immunotherapy for the treatment of hematological malignancies, Hematology 8, 97-104.
Buske, C., Hiddemann, W., and Wormann, B. (1994). Proliferation of human leukemic pre-B cells induced by human bone marrow stromal cells and muline fibroblasts. Cell Biol, Int. 18, 49-1058.
Charbonnier, A., Gaugler, B., Sainty, D., Lafage-Pochitaloff, M., and Olive, D. (1999). Human acute myeloblastic, leukemia cells differentiate in vitro into mature dendritic cells and induce the differentiation of cytotoxic T cells against autologous leukemias. Eur. J. Immunol. 29, 2567-2578.
Choudhury, B. A., Liang, J. C., Thomas, E. K., Flores-Romo, L., Xie, Q. S., Agusala, K., Sutaria, S., Sinha, I., Champlin, R. E., and Claxton, D. F. (1999). Dendritic cells derived in vitro from acute myelogenous leukemia cells stimulate autologous, antileukemic T-cell responses. Blood 93, 780-786.
Cignetti, A., Bryant, E., Allione, B., Vitale, A., Foa, R., and Cheever, M. A. (1999). CD34(+) acute myeloid and lymphoid leukemic blasts can be induced to differentiate into dendritic cells. Blood 94, 2048-2055.
Cignetti, A., Vallario, A., Roato, I., Circosta, P., Allione, B., Casorzo, L., Ghia, P., and Caligaris-Cappio, F. (2004). Leukemia-derived immature dendritic cells differentiate into functionally competent mature dendritic cells that efficiently stimulate T cell responses. J. Immunol. 173, 2855-2865.
Claxton, D. F., McMannis, J., Champlin, R., and Choudhury, A. (2001). Therapeutic potential of leukemia-derived dendritic cells: preclinical and clinical progress. Crit. Rev. Immunol. 21, 147-155.
Collins, A. V., Brodie, D. W., Gilbert, R. J., Iaboni, A., Manso-Sancho, R., Walse, B., Stuart, D. I., van der Merwe, P. A., and Davis, S. J. (2002). The interaction properties of costimulatory molecules revisited. Immunity 17, 201-210.
Corbi, A. L., and Lopez-Rodriguez, C. (1997). CD11c integrin gene promoter activity during myeloid differentiation. Leuk. Lymphoma. 25, 415-425.
Coyle, A. J., and Gutierrez-Ramos, J. C. (2001). The expanding B7 superfamily: increasing complexity in costimulatory signals regulating T cell function. Nat. Immunol. 2, 203-209.
D'Amours, D., Desnoyers, S., D'Silva, I., and Poirier, G. G. (1999). Poly(ADP-ribosyl)ation reactions in the regulation of nuclear functions. Biochem. J. 342, 249-268.
Dührsen, U., and Hossfeld, D. K. (1996). Stromal abnormalities in neoplastic bone marrow diseases. Ann. Hematol. 73, 53-70.
Engelberg, D. (2004). Stress-activated protein kinases-tumor suppressors or tumor initiators? Semin. Cancer Biol. 14, 271-282.
Fujii, S., Fujimoto, K., Shimizu, K., Ezaki, T., Kawano, F., Takatsuki, K., Kawakita, M., and Matsuno, K. (1999). Presentation of tumor antigens by phagocytic dendritic cell clusters generated form human CD34+ hematopoietic progenitor cells: induction of autologous cytotoxic T lymphocytes against leukemic cells in acute myelogenous leukemia patients. Cancer Res. 59, 2150-2158.
Grakoui, A., Bromley, S. K., Sumen, C., Davis, M. M., Shaw, A. S., Allen, P. M., and Dustin, M. L. (1999). The immunological synapse: a molecular machine controlling T cell activation. Science 285, 221-227.
Greenberg, B. R., Woo, L., and Garewal, H. S. (1988). Effects of bone marrow fibroblastic cells and fibroblastic conditioned medium on HL-60 and KG-1. Leuk. Res. 12, 61-66.
Griffin, J. D., and Löwenberg, B. (1986). Clonogenic cells in acute myeloblastic leukemia. Blood 68, 1185-1195.
Hermiston, M. L., Xu, Z., and Weiss, A. (2003). CD45: a critical regulator of signaling thresholds in immune cells. Annu. Rev. Immunol. 21, 107-137.
Hopkins-Donaldson, S., Ziegler, A., Kurtz, S., Bigosch, C., Kandioler, D., Ludwig, C., Zangemeister-Wittke, U., and Stahel, R. (2003). Silencing of death receptor and caspase-8 expression in small cell lung carcinoma cell lines and tumors by DNA methylation. Cell Death Differ. 10, 356-364.
Hubbard, A. K., and Rothlein, R. (2000). Intercellular adhesion molecule-1 (ICAM-1) expression and cell signaling cascades. Free Radic. Biol. Med. 28, 1379-1386.
Igawa, T., Kanda, S., Kanetake, H., Saitoh, Y., Ichihara, A., Tomita, Y., and Nakamura, T. (1991). Hepatocyte growth factor is a potent mitogen for cultured rabbit renal tubular epithelial cells. Biochem. Biophys. Res. Commun. 174, 831-838.
Kankuri, E., Cholujova, D., Comajova, M., Vaheri, A., and Bizik, J. (2005). Induction of hepatocyte growth factor/scatter factor by fibroblast clustering directly promotes tumor cell invasiveness. Cancer Res. 65, 9914-9922.
Kmiecik, T. E., Keller, J. R., Rosen, E., Vande Woude, G. F. (1992). Hepatocyte growth factor is a synergistic factor for the growth of hematopoietic progenitor cells. Blood 80, 2454-2457.
Kubota, T., Nagao, T., and Arimori, S. (1988). Bone marrow fibroblasts-conditioned medium regulates the proliferation of leukemic cells. Tokai J. Exp. Clin. Med. 13, 45-51.
Larsen, S. L., Pedersen, L. O., Buus, S., and Stryhn, A. (1996). T cell responses affected by aminopeptidase N (CD13)-mediated training of major histocompatibility complex class II-bound peptides. J. Exp. Med. 184, 183-189.
Launay, S., Hermine, O., Fontenay, M., Kroemer, G., Solary, E., and Garrido, C. (2005), Vital functions for lethal caspases. Oncogene 24, 5137-5148.
Leist, M., Single, B., Kunstle, G., Volbracht, C., Hentze, H., and Nicotera, P. (1997). Apoptosis in the absence of poly-(ADP-ribose) polymerase. Biochem. Biophys. Res. Commun. 233, 518-522.
Liu, M., Lee, M. H., Cohen, M., Bommakanti, M., and Freedman, L. P. (1996). Transcriptional activation of the Cdk inhibitor p21 by vitamin D3 leads to the induced differentiation of the myelomonocytic cell line U937. Genes Dev. 10, 142-153.
Lotem, J., and Sachs, L. (2002). Cytokine control of developmental programs in normal hematopoiesis and leukemia. Oncogene 21, 3284-3294.
Mangan, J. K., Rane, S. G., Kang, A. D., Amanullah, A., Wong, B. C., and Reddy, E. P. (2004). Mechanisms associated with IL-6-induced up-regulation of Jak3 and its role in monocytic differentiation. Blood 103, 4093-4101.
Marlin, S. D., and Springer, T. A. (1987). Purified intercellular adhesion molecule-1 (ICAM-1) is a ligand for lymphocyte function-associated antigen 1 (LFA-1). Cell 51, 813-819.
Matsumoto, K., Ziober, B. L., Yao, C. C., and Kramer, R. H. (1995). Growth factor regulation of integrin-mediated cell motility. Cancer Metastasis Rev. 14, 205-217.
Mayani, H., Guilbert, L. J., and Janowska-Wieczorek, A. (1992) Biology of the hemopoietic microenvironment. Eur. J. Haematol. 49, 225-233.
Mohty, M., Olive, D., and Gaugler, B. (2002). Leukemic dendritic cells: potential for therapy and insights towards immune escape by leukemic blasts. Leukemia 16, 2197-2204.
Montesano, R., Matsumoto, K., Nakamura, T., and Orci, L. (1991). Identification of a fibroblast-derived epithelial morphogen as hepatocyte growth factor. Cell 67, 901-908.
Nemoto, S., Xiang, J., Huang, S., and Lin, A. (1998). Induction of apoptosis by SB202190 through inhibition of p38beta mitogen-activated protein kinase. J. Biol. Chem. 273, 16415-16420.
Noti, J. D., and Reinemann, B. C. (1995). The leukocyte integrin gene CD11c is transcriptionally regulated during monocyte differentiation. Mol. Immunol. 32, 361-369.
Osugi, Y., Vuckovic, S., and Hart, D. N. (2002). Myeloid blood CD11c(+) dendritic cells and monocyte-derived dendritic cells differ in their ability to stimulate T lymphocytes. Blood 100, 2858-2866.
Platanias, L. C. (2003). Map kinase signaling pathways and hematologic malignancies. Blood 101, 4667-4679.
Rane, S. G., Mangan, J. K., Amanullah, A., Wong, B. C., Vora, R. K., Liebermann, D. A., Hoffman, B., Grana, X., and Reddy, E. P. (2002). Activation of the Jak3 pathway is associated with granulocytic differentiation of mycloid precursor cells. Blood 100, 2753-2762.
Rane, S. G., and Reddy, E. P. (1994). JAK3: a novel JAK kinase associated with terminal differentiation of hematopoietic cells. Oncogene 9, 2415-2423.
Re, F., Arpinati, M., Testoni, N., Ricci, P., Terragna, C., Preda, P., Ruggeri, D., Senese, B., Chirumbolo, G., Martelli, V., et al. (2002). Expression of CD86 in acute myelogenous leukemia is a marker of dendritic/monocytic lineage. Exp. Hematol. 30, 126-134.
Reiss, M., Gamba-Vitalo, C., and Sartorelli, A. C. (1986). Induction of tumor cell differentiation as a therapeutic approach: preclinical models for hematopoietic and solid neoplasms. Cancer Treat. Rep. 70, 201-218.
Roebuck, K. A., and Finmegan, A. (1999). Regulation of intercellular adhesion molecule-1 (CD54) gene expression. J. Leukoc. Biol, 66, 876-888.
Rogalsky, V., Zinzar, S., Golub, E., and Den, T. (1991). Fibroblast factor inducing leukemic cell differentiation and inhibiting malignant cell growth. Ann. N. Y. Acad. Sci. 628, 323-325.
Salvesen, G. S., and Abrams, J. M. (2004). Caspase activation—stepping on the gas or releasing the brakes? Lessons from humans and flies. Oncogene 23, 2774-2784.
Shelley, C. S., Teodoridis, J. M., Park, H., Farokhzad, O. C., Bottinger, E. P., and Amaout, M. A. (2002). During differentiation of the monocytic cell line U-937, Pur alpha mediates induction of the CD11c beta 2 integrin gene promoter. J. Immunol. 168, 3887-3893.
Silzle, T., Randolph, G. J., Kreutz, M., and Kunz-Schughart, L. A. (2004). The fibroblast: sentinel cell and local immune modulator in tumor tissue. Int. J. Cancer 108, 173-180.
Smith, R. S., Smith, T. J., Blieden, T. M., and Phipps, R. P. (1997). Fibroblasts as sentinel cells. Synthesis of chemokines and regulation of inflammation. Am. J. Pathol. 151, 317-322.
Stelzer, G. T., Shults, K. E., and Loken, M. R. (1993). CD45 gating for routine flow cytometric analysis of human bone marrow specimens. Ann. N. Y. Acad. Sci. 677, 265-280.
Tavassoli, M., and Friedenstein, A. (1983). Hemopoietic stromal microenvironment. Am. J. Hematol. 15, 195-203.
Torok-Storb, B., Iwata, M., Graf, L., Gianotti, J., Horton, H., and Byrne, M. C. (1999). Dissecting the marrow microenvironment. Ann. N. Y. Acad. Sci. 872, 164-170.
van Lochem, E. G., van der Velden, V. H. J., te Marvelde, J. G., Westerdaal, N. A. C., and van Dongen, J. J. M. (2004). Immunophenotypic differentiation patterns of normal hematopoiesis in human bone marrow: Reference patterns for age-related changes and disease-induced shifts. Cytometry 60B, 1-13.
Villunger, A., O'Reilly, L. A., Holler, N., Adams, J., and Strasser, A. (2000). Fas ligand, Bcl-2, granulocyte colony-stimulating factor, and p38 mitogen-activated protein kinase: Regulators of distinct cell death and survival pathways in granulocytes. J. Exp. Med. 192, 647-658.
Virag, L., and Szabo, C. (2002). The therapeutic potential of poly(ADP-ribose) polymerase inhibitors. Pharmacol. Rev. 54, 375-429.
Wang, H., and Sullivan, A. K. (1992). Effects of a fibroblast-derived inhibitor on the growth of normal marrow and leukemic clonogenic cells. Leuk. Res. 16, 693-701.
Wang, Z. Y., and Chen, Z. (2000). Differentiation and apoptosis induction therapy in acute promyelocytic leukaemia. Lancet Oncol. 1, 101-106.
Yang, X., Merchant, M. S., Romero, M. E., Tsokos, M., Wexler, L. H., Kontny, U., Mackall, C. L., and Thiele, C. J. (2003). Induction of caspase 8 by interferon gamma renders some neuroblastoma (NB) cells sensitive to tumor necrosis factor-related apoptosis-inducing ligand (TRAIL) but reveals that a lack of membrane TR1/TR2 also contributes to TRAIL resistance in NB. Cancer Res. 63, 1122-1129.
Youn, B. S., Mantel, C., and Broxmeyer, H. E. (2000). Chemokines, chemokine receptors and hematopoiesis. Immunol. Rev. 177, 150-174.
Zheng, P., Wu, Y., Guo, Y., Lee, C., and Liu, Y. (1998). B7-CTLA4 interaction enhances both production of antitumor cytotoxic T lymphocytes and resistance to tumor challenge. Proc. Natl. Acad. Sci. U.S.A. 95, 6284-6289.
Zhu, J., and Emerson, S. G. (2002). Hematopoietic cytokines, transcription factors and lineage commitment. Oncogene 21, 3295-3313.
Zou, W. (2005). Immunosuppressive networks in the tumour environment and their therapeutic relevance. Nat. Rev. Cancer 5, 263-274.
Zuckerman, L. A., Pullen, L., and Miller, J. (1998). Functional consequences of costimulation by ICAM-1 on IL-2 gene expression and T cell activation. J. Immunol. 160, 3259-3268.

1. Method for treating a cell sample, preferably for a diagnostic or therapeutic purpose, said method comprising:
a) culturing mesenchymal cells under conditions that induce the cells to adhere to each other so that multicellular aggregates are formed; and
b) co-culturing the cells in said cell sample with said multicellular aggregates or conditioned medium thereof obtained from step a) in order to regulate the growth and/or status of the cells in said cell sample.

2. The method according to claim 1, wherein in step b) there is a semi-permeable membrane between said cell sample and the multicellular aggregates, said membrane permitting exchange of cytokines and growth factors but separating said mesenchymal cell aggregates from the cells in said cell sample.

3. The method according to claim 1, wherein the cells in said cell sample are leukemia cells.

4. The method according to claim 1 further comprising a step of c) selecting those cells in said cell sample which responded to the co-culturing with said multicellular aggregates.

5. The method according to claim 4, wherein the selected cells responded to the co-culturing by chemotactic movement.

6. The method according to claim 4, wherein said cells are selected for a therapeutic or diagnostic use.

7. The method according to claim 1, wherein said cell sample is a patient sample and said multicellular aggregates are derived from autologous mesenchymal cells.

8. The method according to claim 1, wherein said cell sample is a patient sample and said multicellular aggregates are derived from allogenic mesenchymal cells.

9. A device for selecting cells from a sample of cells, said device comprising a first compartment and a second compartment, said first compartment being arranged within the second compartment, wherein said first compartment comprises multicellular aggregates of fibroblast cells or mesenchymal cells in a buffer, said first compartment being separated from the second compartment by a semi-permeable membrane allowing the exchange of buffer, cytokines and growth factors between the compartments; and wherein the second compartment is separated from the sample of cells by a second membrane having a pore size allowing cells to migrate across the membrane.

10. The device according to claim 9, wherein said compartments and the sample of cells is surrounded by an outer sealing membrane.

11. The device according to claim 9, wherein said semi-permeable membrane does not allow the exchange of cells between said compartments.

12. The device according to claim 9, wherein said semi-permeable membrane has a pore size of 0.2 to 2 μm and said second membrane has a pore size of 3 to 8 μm.

13. The device according to claim 9, wherein said sample is a bone marrow aspirate or peripheral blood sample.

14. Method for treating a sample of leukemia cells, preferably for a diagnostic or therapeutic purpose, said method comprising:
a) culturing fibroblast cells or mesenchymal cells under conditions that induce the cells to adhere to each other so that multicellular aggregates are formed; and
b) co-culturing the leukemia cells in said sample with said multicellular aggregates or conditioned medium thereof obtained from step a) in order to regulate the growth and/or status of the leukemia cells in said sample.
